(19) United States
(12) Patent Application Publication
(10) Pub. No.: US 2005/0098177 A1
(43) Pub. Date: May 12, 2005

(54) Exhalation valve assembly

(76) Inventors: Sajed Haj-Yahya, Taybe (IL); Mark Shahar, Tel-Aviv (IL)

Correspondence Address: DANIEL J SWIRSKY, PO BOX 2345, BEIT SHEMESH, 99544, IL

(21) Appl. No.: 10/704,588

(22) Filed: Nov. 12, 2003

(51) Int. Cl. A61M 16/00

(52) U.S. Cl. 128/204.18; 128/205.24; 128/911

(57) ABSTRACT

Breathing apparatus including an exhalation tube and an outlet port in fluid communication with the exhalation tube, where the outlet port includes an outlet lip that is not uniformly flat in any cutting plane.

### FIELD OF THE INVENTION

The present invention relates to respirators or ventilators in general, and particularly to exhalation valve assemblies therefor.

### BACKGROUND OF THE INVENTION

A ventilated patient who is attached to respirator for assited breathing typically inhales and exhales via separate tubes that merge into Y-connector near the patient. Valves in the inhalation and exhalation tubes open and close at appropriate times to regulate the breathing cycle, with the exhalation valve in the exhalation tube being allowed to open as the patient exhales, while the inhalation valve is simultaneously closed to prevent flow of exhaled gas into the inhalation tube.

Respirator exhalation valves typically include a flexible diaphragm mounted in a valve assembly having an inlet port, an outlet port and a control pressure port. During patient inhalation, the diaphragm rests on a valve seat and prevents gas in the exhalation tube from circulating back towards the patient via the inlet port, while during exhalation the diaphragm lifts from the valve seat and allows exhaled gas to flow from inlet port through the outlet port. The pressure control port allows a control pressure to be applied to the diaphragm from above, ensuring that the diaphragm remains firmly seated during inhalation. The control pressure is typically sufficiently reduced during exhalation to allow the diaphragm to be unseated by patient expiration pressure.

Should the outlet port become blocked, lung over-pressurization may occur, leading to patient injury or death. An exhalation valve assembly that prevents outlet port blockage would therefore be advantageous.

### SUMMARY OF THE INVENTION

In accordance with a preferred embodiment of the present invention, an exhalation valve assembly is provided with an improved outlet port that prevents blockage thereof.

### BRIEF DESCRIPTION OF THE DRAWINGS

The present invention will be understood and appreciated more fully from the following detailed description taken in conjunction with the appended drawings in which:

FIG. 1 is a simplified pictorial illustration of patient breathing apparatus and exhalation valve assembly, constructed and operative in accordance with a preferred embodiment of the present invention;

FIG. 2 is a simplified cross-sectional illustration of an exhalation valve assembly 200, constructed and operative in accordance with a preferred embodiment of the present invention; and

FIGS. 3A-4C are simplified pictorial illustrations of exhalation valve assembly outlet configurations, constructed and operative in accordance with a preferred embodiment of the present invention.

### DETAILED DESCRIPTION OF PREFERRED EMBODIMENTS

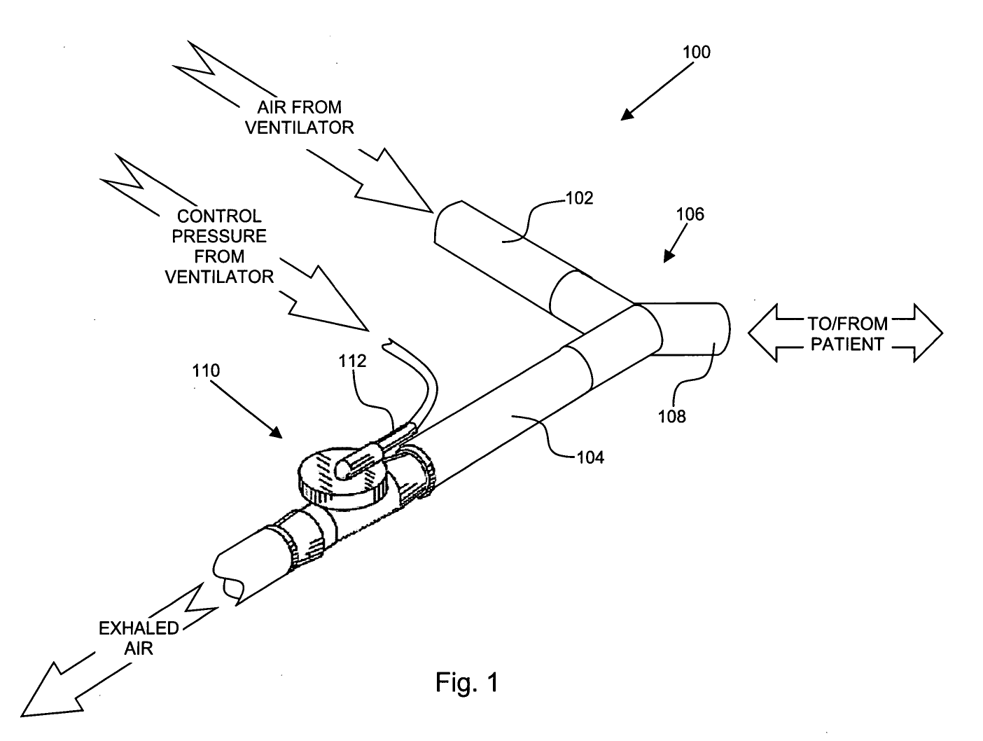

Reference is now made to FIG. 1 which is a simplified pictorial illustration of patient breathing apparatus and exhalation valve assembly, constructed and operative in accordance with a preferred embodiment of the present invention. As shown in FIG. 1, patient breathing apparatus 100 typically includes an inhalation tube 102 connected to a ventilator (not shown), an exhalation tube 104, and a Y-connector 106, to which both the inhalation and exhalation tubes are connected. Y-connector 106 is shown having a patient connector 108 through which the patient breathes. An exhalation valve assembly 110 is connected to exhalation tube 104 and is controlled by a control pressure line 112 connected to exhalation valve assembly 110.

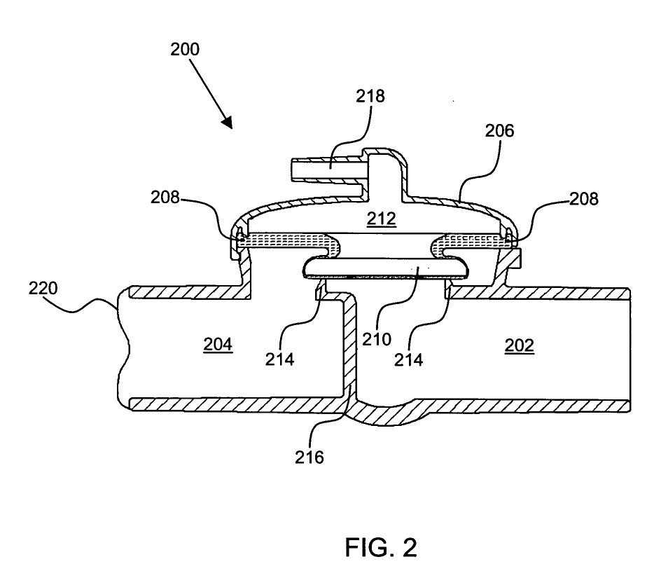

Reference is now made to FIG. 2 which is a simplified cross-sectional illustration of an exhalation valve assembly 200, constructed and operative in accordance with a preferred embodiment of the present invention. As shown in FIG. 2, exhalation valve assembly 200 includes an inlet port 202, such as for connection to exhalation tube 104 (FIG. 1), and an outlet port 204, ports 202 and 204 typically being oriented on a common axis. Exhalation valve assembly 200 further includes a valve housing 206 covering the junction of ports 202 and 204. A gasket 208 preferably forms a seal between housing 206 and ports 202 and 204, with gasket 208 contacting a diaphragm 210 on its surface facing housing 206 to define a valve chamber 212. Diaphragm 210 is supported by a valve seat 214 formed by the wall of inlet port 202 and the terminus of a divider 216 that separates ports 202 and 204. Housing 206 also includes a control pressure port 218 in fluid communication with chamber 212.

When control pressure is applied to chamber 212 above the diaphragm exceeds the pressure in inlet port 202, diaphragm 210 is held against valve seat 214, preventing exhalation flow from inlet port 202 to outlet port 204. When the control pressure is removed or sufficiently reduced, diaphragm 210 may be lifted from valve seat 214 by patient expiration pressure, allowing exhalation flow.

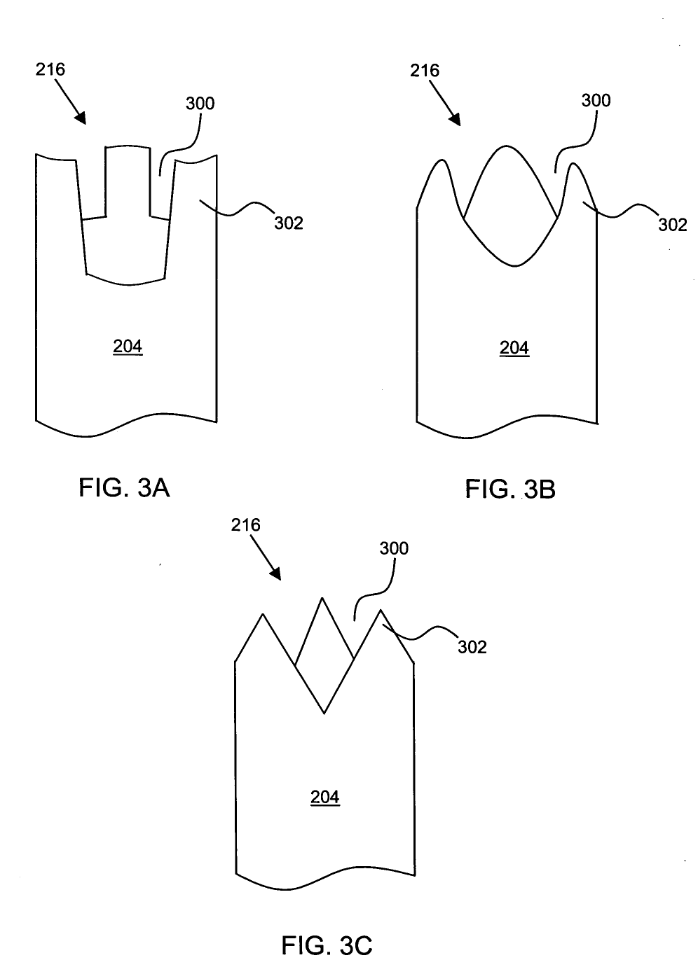

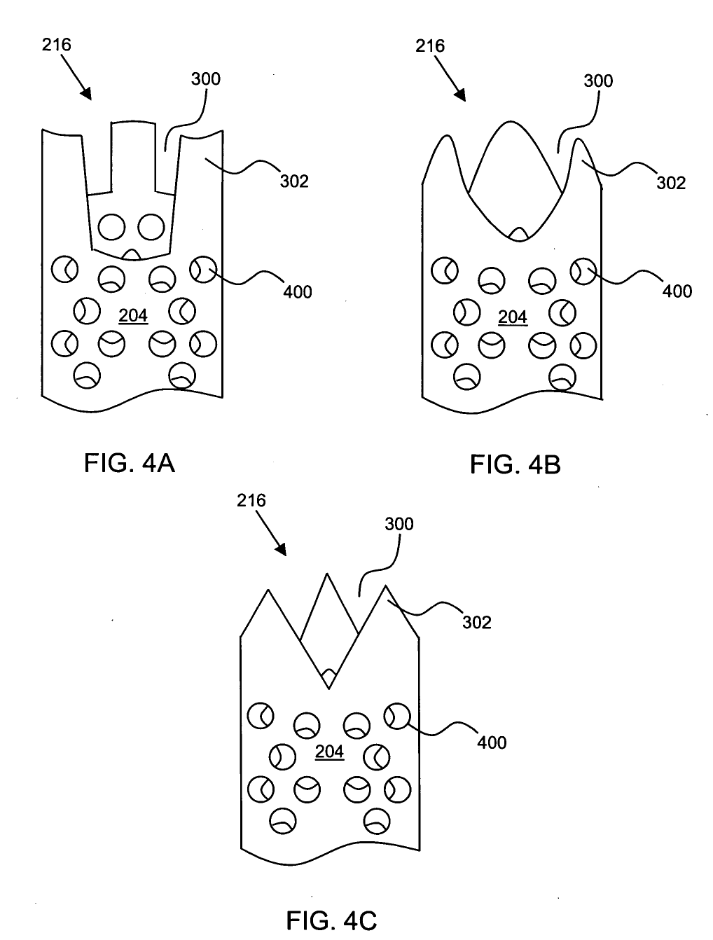

To prevent blockage of outlet port 204 during patient exhalation, port 204 preferably includes an outlet lip 220 that is not uniformly flat in any cutting plane. For example, outlet lip 220 may be crenelated, as may be seen in greater detail in FIGS. 3A-3C , where outlet lip 220 has one or more crenels 300 and merlons 302 that may be rectangular (FIG. 3A), rounded (FIG. 3B), saw-toothed (FIG. 3C), or otherwise shaped such that outlet lip 216 could abut a flat surface, such as a wall or floor, and still permit gas flow through its crenels. Additionally or alternatively, outlet port 204 may have one or more apertures 400 formed through its wall for like effect, as may be seen in greater detail in FIGS. 4A-4C.

While the present invention has been described with reference to one or more specific embodiments, the description is intended to be illustrative of the invention as a whole and is not to be construed as limiting the invention to the embodiments shown. It is appreciated that various modifications may occur to those skilled in the art that, while not specifically shown herein, are nevertheless within the true spirit and scope of the invention.

1. Breathing apparatus comprising:
an exhalation tube; and
an outlet port in fluid communication with said exhalation tube, wherein said outlet port includes a crenelated outlet lip.

2. Breathing apparatus according to claim 1 wherein said outlet lip comprises at least one rectangular merlon.

3. Breathing apparatus according to claim 1 wherein said outlet lip comprises at least one rounded merlon.

4. Breathing apparatus according to claim 1 wherein said outlet lip comprises at least one saw-toothed merlon.

5. Breathing apparatus according to claim 1 wherein said outlet port comprises a wall having at least one aperture formed therein.

6. Breathing apparatus according to claim 1 and further comprising an exhalation valve intermediate said exhalation tube and said outlet port.

7. Breathing apparatus according to claim 6 wherein said exhalation valve is in fluid communication with said exhalation tube via an inlet port.

8. Breathing apparatus according to claim 6 wherein said exhalation valve is in fluid communication with said outlet port.

9. Breathing apparatus according to claim 1 wherein said outlet port comprises a wall having at least one aperture formed therein.

10. Breathing apparatus comprising:
an exhalation tube; and
an exhalation valve in fluid communication with said exhalation tube via an inlet port and having an outlet port, wherein said outlet port includes a crenelated outlet lip.

11. Breathing apparatus according to claim 10 wherein said outlet lip comprises at least one rectangular merlon.

12. Breathing apparatus according to claim 10 wherein said outlet lip comprises at least one rounded merlon.

13. Breathing apparatus according to claim 10 wherein said outlet lip comprises at least one saw-toothed merlon.

14. Breathing apparatus according to claim 10 wherein said outlet port comprises a wall having at least one aperture formed therein.

15. Breathing apparatus comprising:
an exhalation tube; and
an outlet port in fluid communication with said exhalation tube, wherein said outlet port includes an outlet lip that is not uniformly flat in any cutting plane.

16. Breathing apparatus according to claim 15 wherein said outlet port comprises a wall having at least one aperture formed therein.

17. Breathing apparatus according to claim 15 and further comprising an exhalation valve intermediate said exhalation tube and said outlet port.

18. Breathing apparatus according to claim 17 wherein said exhalation valve is in fluid communication with said exhalation tube via an inlet port.

19. Breathing apparatus according to claim 17 wherein said exhalation valve is in fluid communication with said outlet port.

20. Breathing apparatus according to claim 15 wherein said outlet port comprises a wall having at least one aperture formed therein.
